(19) United States
(12) Patent Application Publication
(10) Pub. No.: US 2022/0175631 A1
(43) Pub. Date: Jun. 9, 2022

(54) COSMETIC COMPOSITION COMPRISING A HYDROXYALKYL STARCH AS MAIN EMULSIFYING SYSTEM

(71) Applicant: CHANEL PARFUMS BEAUTE, NEUILLY SUR SEINE (FR)

(72) Inventors: Hélène DE CLERMONT GALLERANDE, PANTIN CEDEX (FR); Servane COROLLER, PANTIN CEDEX (FR)

(73) Assignee: CHANEL PARFUMS BEAUTE, NEUILLY SUR SEINE (FR)

(21) Appl. No.: 17/545,886

(22) Filed: Dec. 8, 2021

(30) Foreign Application Priority Data
Dec. 8, 2020 (FR) 2012829

(51) Int. Cl. A61K 8/06; A61K 8/73; A61K 8/92; A61K 8/81; A61Q 1/10

(52) U.S. Cl. CPC A61K 8/062; A61K 8/732; A61K 8/92; A61K 2800/591; A61K 8/8135; A61Q 1/10; A61K 2800/30; A61K 8/8111

(57) ABSTRACT

A cosmetic composition in particular for coating keratinous fibers in the form of a wax-in-water emulsion and including at least one hydroxyalkyl starch, characterized in that the hydroxyalkyl starch constitutes the main emulsifying system of the composition. Also, a process for makeup and/or non-therapeutic care in particular for keratinous fibers, including the application of a composition such as previously described on the keratinous fibers. Further, the use of a composition such as previously described in order to get a homogeneous and/or building makeup of the eyelashes on the keratinous fibers, in particular eyelashes.

### FIELD OF THE INVENTION

The present disclosure relates to the field of cosmetic formulation, and in particular the formulation of mascaras.

### DESCRIPTION OF RELATED ART

Among the mascara formulations, mascaras called “emulsion mascaras” or “washable mascara” are in particular known; they are made up of a wax or mixture of waxes dispersed by means of at least one surfactant and an aqueous phase, further containing hydrosoluble polymers and also pigments.

These solid particles, and in particular the waxes, are generally dispersed by means of a surfactant system. The choice of the surfactant system is preponderant in obtaining a stable dispersion, insofar as the surfactants play an important role at the interface in the interactions between wax particles within the formula.

The surfactant system traditionally used is composed of triethanolamine stearate or derivatives thereof.

In fact, this surfactant system serves to preserve the flexibility of the paste formed, to provide a good deposit on the eyelashes and a volume lasting throughout the day. Triethanolamine stearates and derivatives are excellent surfactants for mascara compositions and substitutions are therefore difficult.

However, primary, secondary and tertiary alkanolamines, which includes triethanolamine stearates and derivatives, are responsible for the formation of nitrosamines. These nitrosamines are impurities that are entirely undesirable in cosmetic compositions because of their toxicity.

The substitution of triethanolamine stearates and derivatives is therefore currently a major concern of formulators in the cosmetic field.

There are compositions having sucrose ester based surfactant systems substituted for the triethanolamine stearates and derivatives. However, the sucrose esters have the disadvantage of not providing volume on the eyelashes and only very discrete makeup results are achieved by this type of formulation. A volumizing mascara is therefore not conceivable with this type of emulsifying system.

Consequently, a need remains for providing cosmetic compositions for coating keratinous fibers, in particular eyelashes, free of triethanolamine or derivatives thereof, and having a satisfactory building power, with which in particular to get a thick makeup of the keratin fibers, in particular eyelashes, again called loading makeup.

### BRIEF SUMMARY OF THE INVENTION

The object of the present invention is specifically to respond to this need.

Surprisingly and unexpectedly, the inventors of the present application resolved this problem by means of a cosmetic composition, in particular for coating keratinous fibers, more specifically eyelashes, having the form of a wax-in-water emulsion and comprising at least one hydroxyalkyl starch as main emulsifying system.

More precisely, the inventors were able to observe that the emulsifying system based on at least one hydroxyalkyl starch defined in the present application made it possible to get a stable dispersion of a large quantity of waxes, comparable to the dispersion obtained with a triethanolamine stearate based emulsification system.

In the sense of the present invention, the term “keratinous fibers” covers hair, eyelashes, eyebrows and also extends to synthetic extensions and false eyelashes.

A “wax-in-water emulsion” is understood in the present invention to mean a composition comprising at least one wax or mixture of waxes dispersed in a continuous aqueous phase by means of at least one surfactant.

“Stable” is understood in the present invention to mean a composition which, after having been placed in an oven at 20° C. for 2 months, does not have grains perceptible to the touch when the thin film of the composition is sheared between fingers.

Thus, according to one of the aspects thereof, the invention relates to a cosmetic composition for coating keratinous fibers in the form of a wax-in-water emulsion comprising at least one first emulsifying system, called main emulsifying system, optionally a second emulsifying system, called secondary emulsifying system, characterized in that said main emulsifying system consists of a hydroxyalkyl starch.

Advantageously, a composition conforming to the invention may comprise under 1%, preferably under 0.5% by weight of triethanolamine or derivatives thereof, and better is free of triethanolamine or derivatives thereof. This element does not constitute either the main emulsifying system, or the secondary emulsifying system.

Particularly advantageously, a composition from the invention may comprise a concentration of waxes ranging from 0.1% to 50%, preferably from 1% to 40%, more preferably from 5% to 30% by weight relative to the total weight of the composition.

Because of the high concentration of waxes that it may incorporate, a composition according to the invention thus has a sufficiently thick texture to get a building and volumizing deposit on the eyelashes.

The compositions from the invention are particularly suited for makeup for eyelashes, good in particular for easy application on the eyelashes and also getting a smooth and homogeneous deposit.

According to another of the aspects thereof, the invention relates to a process for makeup and/or non-therapeutic care for keratinous fibers, in particular eyelashes, comprising the application of a composition such as previously described on said keratinous fibers.

The present invention also targets the use of a composition such as previously described in order to get a homogeneous and/or buildable makeup of the eyelashes on the keratinous fibers, in particular eyelashes.

Finally, the object of the invention is the use of hydroxypropyl starch phosphate as single emulsifying system for a mascara cosmetic composition.

The term “buildable” is understood in the present invention to describe a thick makeup for eyelashes.

Other features, properties and advantages of the present invention will appear more clearly upon reading the following description and examples.

Main Emulsifying System

Hydroxyalkyl starch constitutes the main emulsifying system of the composition.

“Main emulsifying system” is understood to mean a system which on its own serves to stabilize an emulsion. Thus, in their absence, the stability of the emulsion cannot be obtained.

Advantageously, the hydroxyalkyl starch constitutes the only emulsifying system of the composition.

Additionally, the inventors remarked that the use of hydroxyalkyl starch as main surfactant system made it possible to greatly reduce the percentage of thickener used in the formula. The hydroxyalkyl starch gels the composition, giving it both an emulsifying role and also a thickening role. Its use also serves to limit the number of ingredients used for the formulation of a mascara.

“Only” designates a complete absence of any other surfactant system. Thus, a composition which has a hydroxyalkyl starch as its only emulsifying system therefore has a single main emulsifying system and does not comprise a secondary emulsifying system nor triethanolamine or derivatives thereof in the composition thereof.

The hydroxyalkyl starch according to the invention may be selected from hydroxyethyl starch, hydroxypropyl starch, and in particular hydroxypropyl starch phosphate and hydroxyethyl starch phosphate or a mixture of these compounds.

Advantageously, the hydroxyalkyl starch is the hydroxypropyl starch phosphate. This starch is in particular sold by Asko Noble under the names Structure® ZEA and Structure® XL.

The hydroxyalkyl starch may be present in the composition at a concentration ranging from 2% to 10% by weight, preferably from 4% to 8% by weight and better around 5% by weight relative to the total weight of the composition.

In fact, the best compromises between stability and texture of the formula are obtained in these hydroxyl alkyl starch concentrations. A starch concentration which is too low could change the stability of the composition, whereas a concentration that is too high generates too much sticking of the composition.

Secondary Emulsifying System

The cosmetic composition for coating keratinous fibers may comprise in addition to the main emulsifying system, a secondary emulsifying system.

“Secondary emulsifying system” is understood to mean a system which comes to complete the main emulsifying system but which alone is not sufficient for getting a stable emulsion because of the nature or quantity thereof.

The secondary emulsifying system may be composed of any emulsifier known in the state-of-the-art, except for triethanolamine or one of the derivatives thereof. The emulsifiers used in the secondary emulsifying system may be nonionic, anionic, cationic or amphoteric emulsifiers. In particular one can refer to document “Encyclopedia of Chemical Technology, KIRK-OTHMER,” volume 22, third edition, 1979, WILEY.

The composition according to the invention of course includes a physiologically acceptable medium.

In the meaning of the present application, “physiologically acceptable compound or medium” is understood to mean a compound or medium whose use is compatible with application on eyelashes.

Aqueous Phase

The composition according to the invention comprises an aqueous phase which forms the continuous phase of the composition.

Composition with continuous aqueous phase is understood to mean that the composition has a conductivity, measured at 25° C., greater than or equal to 23 μS/cm (microSiemens/cm), where the connectivity is measured, for example, by using a Mettler Toledo MPC227 conductivity meter and an Inlab730 conductivity measurement cell. The measurement cell is submerged in the composition, so as to eliminate air bubbles which could form between the two electrodes of the cell. The connectivity is read once the value from the connectivity meter stabilizes. An average is done after three successive measurements.

The aqueous phase comprises water and at least one water-soluble solvent. In the present invention, “water-soluble solvent” designates a compound that is liquid at ambient temperature and miscible in water (miscibility in water over 50% by weight at 25° C. and atmospheric pressure). Hydrosoluble solvents which may be used in compositions according to the invention may further be volatile. Among the hydrosoluble solvents which may be used in compositions conforming to the invention, the following can in particular be listed: lower mono-alcohols having 1 to 5 carbon atoms such as ethanol and isopropanol, glycols having 2 to 8 carbon atoms such as ethylene glycol, propylene glycol, 1,3-butylene glycol and dipropylene glycol, C₃-C₄ ketones and C₂-C₄ aldehydes.

The aqueous phase (water and possibly the solvent miscible in water) is generally present in the composition according to the present application at a concentration ranging from 1% to 95% by weight, relative to the total weight of the composition, preferably ranging from 3% to 80% by weight, and preferably ranging from 5% to 60% by weight.

In the composition conforming to the invention, the total concentration of surfactants may range from 1% to 13% by weight relative to the total weight of the composition, preferably from 3% to 10% and better around 5% by weight.

According to an embodiment, the cosmetic composition according to the present application comprises less than 1%, preferably less than 0.5% by weight of triethanolamine, and better, is free of triethanolamine.

According to a variant, the composition according to the present application comprises less than 1%, preferably less than 0.5% by weight of triethanolamine, and better, is free of triethanolamine.

Wax(es)

The composition according to the present application comprises at least one wax.

Wax is understood in the present invention to mean a lipophilic compound, soluble at ambient temperature (25° C.), reversibly changing from solid to liquid state, having a melting point greater than or equal to 30° C. which may range up to 120° C. The melting point of the wax may be determined by using a differential scanning calorimeter (DSC), for example the calorimeter sold by Metler under the name DSC 30.

The waxes may be hydrocarbon, fluorine and/or silicone based and be of vegetable, mineral, animal and/or synthetic origin. In particular, the waxes have a melting point over 25° C. and better over 45° C.

The wax may be present at a concentration ranging from 0.1% to 50% by weight relative to the total weight of the composition, better from 1% to 40% and still better from 5% to 30% by weight.

In particular, hydrocarbon waxes can be used such as: beeswax, lanolin wax and Chinese insect waxes; rice wax, carnauba wax, candelilla wax, ouricurry wax, alfa wax, cork fiber wax, sugarcane wax, Japanese wax and sumac wax; Montan wax, microcrystalline waxes, paraffins and ozokerite; polyethylene waxes, waxes obtained by Fischer-Tropsch synthesis and waxy copolymers along with esters thereof.

Waxes obtained by catalytic hydrogenation of animal or vegetable oils having C₈-C₃₂, linear or branched, fatty chains may also be listed. Among these, the following in particular may be listed: hydrogenated jojoba oil, isomerized jojoba oil such as trans isomerized partially hydrogenated jojoba oil made or sold by Desert Whale under the commercial name ISO-JOJOBA-50®, hydrogenated sunflower oil, hydrogenated castor oil, hydrogenated copra oil and hydrogenated lanolin oil, di-(1,1,1 trimethylol propane) tetrastearate sold under the name Hest 2T-4S″ by Heterene, di-(1,1,1-trimethylol propane) tetrabehenate sold under the name Hest 2T-4B by Heterene. Silicone waxes such as alkyl or alkoxy-dimethicone with 16 to 45 carbon atoms, and fluorine-based waxes may even be cited.

Waxes obtained by hydrogenation of olive oil esterified with stearylic alcohol sold under the name “Phytowax Olive 18 L 57” or even waxes obtained by hydrogenation of castor oil esterified with cetylic alcohol sold under the name “Phytowax Castor 16L73” by Sophim may even be used. Such waxes are described in the application FR-A-2,792,190.

According to a specific embodiment, the compositions conforming to the invention may comprise at least one wax called sticky wax, meaning having a stickiness greater or equal to 0.7 N·s and a hardness less than or equal to 3.5 MPa. The use of a sticky wax may in particular make it possible to obtain a cosmetic composition which is easy to apply on the eyelashes since it has a good hold on the eyelashes and which leads to a smooth homogeneous and thickening makeup.

The sticky wax used may have in particular a stickiness ranging from 0.7 N·s to 30 N·s, in particular greater than or equal to 1 N·s, in particular ranging from 1 N·s to 20 N·s, in particular greater than or equal to 2 N·s, in particular ranging from 2 N·s to 10 N·s, and in particular from 2 N·s to 5 N·s.

The stickiness of the wax is determined by the measurement of the change of the force (compressive force or stretching force) as a function of time, at 20° C., by means of a texture analyzer sold by Rheo under the name “TA-TX2i®” equipped with a probe of acrylic polymer shaped like a cone forming a 45° angle. The following is the measurement protocol:
The wax is melted at a temperature equal to the melting point of the wax+10° C. The melted wax is poured into a container 25 mm in diameter and 20 mm deep.
The wax is recrystallized at ambient temperature (25° C.) for 24 hours such that the surface of the wax is flat and smooth, and then the wax is stored for at least one hour at 20° C. before measuring the stickiness.
The texture analyzer probe is moved at a speed is 0.5 mm/s, and then enters into the wax to a penetration depth of 2 mm. When the probe has penetrated the wax to a depth of 2 mm, the probe is held steady for one second (corresponding to the relaxation time) and is then withdrawn at a speed of 0.5 mm/s.
During the relaxation time, the force (compression force) decreases greatly until becoming zero and then, during withdrawal of the probe, the force (stretching force) becomes negative and then again increases to the value 0. The stickiness corresponds to the integral of the force curve as a function of time for the part of the curve corresponding to the negative values of the force (stretching force). The stickiness value is expressed in N·s.

The sticky wax that may be used generally has a hardness less than or equal to 3.5 MPa, in particular ranging from 0.01 MPa the 3.5 MPa, in particular ranging from 0.05 MPa to 3 MPa, or even ranging from 0.1 MPa to 2.5 MPa.

The hardness is measured according to the protocol previously described.

The following can be used as sticky wax: C₂₀-C₄₀ alkyl (hydroxystearyloxy)stearate (with the alkyl group comprises 20 to 40 carbon atoms), alone or as a mixture, in particular a C₂₀-C₄₀ alkyl 12-(12′-hydroxystearyloxy)stearate.

Such a wax is in particular sold under the names “Kester Wax K 82 P®” and 5 “Kester Wax K 80 P®” by Koster Keunen.

The waxes listed above generally have a melting point starting below 45° C.

It is also possible to use microcrystalline wax sold under the reference SP18 by Strahl and Pitsch which has a hardness of about 0.46 MPa and the stickiness value of about 1 N·s.

The one or more waxes may be present in a wax aqueous microdispersion form. Wax aqueous micro dispersion is understood to mean an aqueous dispersion of wax particles, in which the size of said wax particles is less than or equal to about 1 pm.

Wax micro dispersions are stable dispersions of wax colloidal particles and are in particular described in “Microemulsions Theory and Practice,” L. M. Prince Ed. Academic Press (1977) pages 21-32.

In particular, these wax microdispersions may be obtained by melting the wax in the presence of a surfactant, and possibly a portion of water, and then progressively adding hot water with stirring. The intermediate formation of a water-in-oil type emulsion is observed, followed by a phase inversion finally resulting in an oil-in-water type microemulsion. On cooling, a stable microdispersion of solid colloidal wax particles results.

The wax microdispersions can also be obtained by stirring of the wax, surfactant and water mixture by stirring means such as ultrasound, high-pressure homogenizer and turbines.

The particles in the wax microdispersion preferably have average dimensions below 1 pm (in particular ranging from 0.02 pm to 0.99 pm), preferably below 0.5 pm (in particular ranging from 0.06 pm to 0.5 pm).

These particles are made up essentially of one wax or of a mixture of waxes.

They may just the same comprise a minority proportion of oily or pasty fat additives, a surfactant and/or a usual light post soluble additive/active ingredient.

Film-Forming Polymer

The compositions according to the present application may also contain at least one hydrophilic or lipophilic film-forming polymer.

“Film-forming polymer” is understood in the present application to mean a polymer able on its own or in the presence of an auxiliary filming agent to form a film that is microscopically continuous and adhering to the eyelashes, and preferably a cohesive film, and better yet a film whose cohesion and mechanical properties are such that said film can be isolated and handled in isolation, for example when said film is made by casting on a nonstick surface like a Teflon or silicone coated surface.

Generally, the “film-forming polymer” concentration of the compositions according to the present application go from 0.1 to 40%, preferably from 0.5 to 30%, better from 1 to 20% by weight relative to the total weight of the composition.

The hydrophilic film-forming polymer may be a hydrosoluble polymer or come as a dispersion in an aqueous medium.

The following can be listed among the film-forming polymers that can be used in the composition from the present invention: synthetic polymers of radical or polycondensate type, polymers of natural origin, and mixtures thereof.

The following can be listed as examples of hydrosoluble film-forming polymers:
proteins like vegetable proteins such as wheat and soy proteins; animal proteins such as keratin, for example keratin hydrolyzates and sulfonated keratin;
cellulose polymers such as hydroxyethylcellulose, hydroxypropylcellulose, methylcellulose, ethylhydroxyethylcellulose, carboxymethylcellulose, and also quaternized derivatives of cellulose;
acrylic polymers or copolymers such as polyacrylates or polymethacrylates;
vinyl polymers such as polyvinylpyrrolidones, copolymers of methylvinyl ether and malicanhydride, copolymer of vinyl acetate and crotonic acid, copolymers of vinylpyrrolidone and vinyl acetate; copolymers of vinylpyrrolidone and caprolactam; polyvinyl alcohol;
anionic, cationic, amphoteric or nonionic chitin or chitosan polymers;
gum arabic, guar gum, xanthan derivatives and karaya gum;
alginates and carrageenans;
glycosaminoglycans, hyaluronic acid and derivatives thereof;
shellac resin, sandarac gum, dammars, elemis, copals;
deoxyribonucleic acid;
mucopolysaccharides such as chondroitin sulfate, and mixtures thereof;
natural polysaccharides such as those produced from starch by fermentation using a natural yeast such as Pullulan® sold by Hayashibara International, or galactoarabinan sold under the name Lara care A200® by Larex;
oligosaccharide mixtures such as inuline sold under the name Inutec H25P® by Naturochim.

The film-forming polymer may also be present in the composition in the form of particles dispersed in an aqueous phase, generally known under the name of latex or pseudolatex. The techniques for preparing these dispersions are well known to the person skilled in the art.

The following may be used as aqueous dispersion of film-forming polymer: acrylic dispersions sold under the names Neocryl XK-90®, Neocryl A-1070®, Neocryl A-1090®, Neocryl BT-62®, Neocryl A-1079® and Neocryl A-523® by Avecia-Neoresins, Dow Latex 432® by Dow Chemical, Daitosol 5000 AD® or Daitosol 5000 SJ® by Daito Kasey Kogyo; Syntran 5760® by Allianz Opt® by Rohm and Haas, or even aqueous dispersions of polyurethane sold under the names Neorez R-981® and Neorez R-974® by Avecia-Neoresins, Avalure UR-405®, Avalure UR-410®, Avalure UR-425®, Avalure UR-450®, Sancure 875®, Avalure UR-445® and Sancure 2060® by Noveon, Impranil 85®, BaycusanC1001®, BaycusanC1004® by Bayer, Aquamere H-1511® by Hydromer; sulfopolyesters sold under the trade name Eastman AQ® by Eastman Chemical Products, vinyl dispersion such as Mexomère PAM®, aqueous dispersions of polyvinyl acetate like “Finalbrand®” from Nisshin Chemical or those sold by Union Carbide, aqueous dispersions of terpolymers such as vinyl pyrrolidone, dimethylaminopropyl methacrylamide and lauryldimethylpropylmethacrylamidoammonium chloride such as Styleze W from ISP, aqueous dispersions of polyurethane/polyacrylic hybrid polymers such as those sold under the name “Hybridur®” by Air Products or “Duromer®” from National Starch, the core/shell type dispersions: for example those sold by Ato Fina under the name Kynar (core: fluoro; shell: acrylic) or even those described in the document U.S. Pat. No. 5,188,899 (core: silica; shall: silicone) and mixtures thereof.

The lipophilic polymer may be in solution or dispersed in a nonaqueous solvent phase or even dispersed in a mixture of waxes. The lipophilic polymer may in particular be an ethylene/vinyl acetate copolymer.

It is generally present in the composition in sufficient quantity to avoid the formation of clumps during application of the formula on the eyelashes.

The quantity of ethylene/vinyl acetate copolymer could thus be adjusted based on the level of waxes present in the composition from the invention.

The ratio by weight of ethylene/vinyl acetate copolymer to wax or mixture of waxes in the composition from the invention is preferably included between 1:55 and 1:9.

Preferably the ratio by weight of ethylene/vinyl acetate copolymer to wax or mixture of waxes in the composition from the invention is included between 1:55 and 1:15.

Even more preferably, the ratio by weight of ethylene/vinyl acetate copolymer to wax or mixture of waxes in the composition from the invention is included between 1:50 and 1:20.

In a preferred embodiment of the invention, an ethylene/vinyl acetate copolymer previously dispersed in a mixture of waxes is used.

This has the advantage of reducing the melting point of the copolymer, which makes it possible to reduce the heating temperature for the preparation of the composition from the invention.

The waxes used to pre-disperse the copolymer from the invention may be selected from those which are mentioned below. Preferably, the waxes used for pre-dispersing the polymer are selected from synthetic wax, microcrystalline wax or mixture thereof.

In particular, a pre-mixture of copolymer in waxes comprising 80% by weight of ethylene/vinyl acetate copolymer can be used in which the vinyl acetate percentage is 13% by weight relative to the total weight of the copolymer, where the copolymer is pre-dispersed in a mixture of waxes comprising 15% by weight of microcrystalline wax, and 5% by weight of synthetic wax sold under the name Cérylène B72® by Baerlocher.

Thickeners

The compositions according to the present application may also contain at least one, preferably hydrophilic, thickener compound which may be selected from:
acrylic or methacrylic acid homo- or co-polymers or salts or esters thereof and in particular the products sold under the name Versicol F® or Versicol K® by Allied Colloid, Utrahold 80 by Ciba-Geigy, SYNTHALEN K type polyacrylic acids;
acrylic acid and acrylamide copolymers sold in sodium salt form under the name Reten® by Hercules, the sodium salts of polyhydroxycarboxylic acids sold under the name Hydagen F® by Henkel;
Pemulen type polyacrylic acid/alkyl acrylate copolymers;
AMPS (acrylamidomethylpropane sulfonic acid polymer, partially neutralized with ammonia and highly crosslinked) sold by Clariant;
Sepigel® or Simulgel® type AMPS/acrylamide copolymers sold by Seppic; AMPS/polyoxyethylene alkyl methacrylate copolymers (crosslinked or non-), and mixtures thereof;
associative polyurethanes such as the C 16 -0E 120 -C 16 polymer from Servo Deldon (sold under the name SER AD FX1100, urethane function molecule and 1300 mean molecular weight), where OE is an oxyethylene unit, Rheolate 205 with urea function sold by Rheox or even Rheolate 208 or 204 (these polymers are sold in pure form) or DW 1206B from Rohm & Haas with C20 alkyl chain and urethane bond, sold as 20% active material in water.

Solutions or dispersions of these associative polyurethanes, in particular in water or in hydroalcoholic medium can also be used. The following may be given as examples of such polymers: SER AD fx1010, SER AD FX1035 and SER AD 1070 from Servo Delden, Rheolate 255, Rheolate 278 and Rheolate 244 sold by Rheox. The product DW 1206F and DW 1206J, and also Acrysol RM 184 or Acrysol 44 from RHOM & HAAS, or even Borchigel LW 44 from Borch ers and mixtures thereof can also be used.

Some hydrosoluble film-forming polymers listed above may also play the role of hydrosoluble thickener.

According to a specific embodiment, the cosmetic composition according to the present application comprises less than 1%, preferably less than 0.5% by weight of thickening compounds, and better, is free of thickening compounds. Effectively, since hydroxyalkyl starch implemented in the present application has thickening properties, it is generally not necessary to add other thickeners in order to guarantee getting a stable emulsion having the texture sought.

It is all the same possible to add thickeners according to the texture intended for the composition according to the invention. If they are present in the invention, the hydrophilic thickeners may be added at a concentration ranging from 0.05 to 40% by weight relative to the total weight of the composition, preferably from 0.1 to 20% and better from 0.5 to 15% by weight.

Oils

The compositions according to the present application may also contain at least one or more oils or organic solvent.

Oil or organic solvent is understood to mean a nonaqueous body liquid at ambient temperature and atmospheric pressure. The oil may be volatile or nonvolatile.

“Volatile oil or organic solvent” is understood in the present invention to mean any nonaqueous medium which could evaporate on contact with keratinous materials in less than one hour at ambient temperature and atmospheric pressure. The one or more volatile organic solvents and volatile oils from the invention are organic solvents and volatile cosmetic oils, liquid at ambient temperature, having, at ambient temperature and atmospheric pressure, a nonzero vapor pressure ranging from 0.13 Pa to 40,000 Pa (10⁻³ to 300 mmHg), in particular ranging from 1.13 Pa to 13,000 Pa (0.01 to 100 mmHg) and more particularly ranging from 1.3 Pa to 1300 Pa (0.01 to 10 mmHg).

“Nonvolatile oil” is understood to mean an oil remaining on keratinous materials at ambient temperature and atmospheric pressure at least several hours and having in particular a vapor pressure below 10⁻³ mmHg (0.13 Pa).

The oil may be present in the composition at a concentration ranging from 0.05 to 30%, preferably 0.1 to 15% by weight relative to the total weight of the composition. The composition according to the invention may comprise volatile oils and/or nonvolatile oils, and mixtures thereof.

The volatile oils (or organic solvents) may be hydrocarbon oils, silicone oils, fluorine oils or mixtures thereof.

“Hydrocarbon oil” is understood to mean an oil containing mainly hydrogen and carbon atoms and possibly oxygen, nitrogen, sulfur and phosphorus atoms. Volatile hydrocarbon oils may be selected from hydrocarbon oils having 8 to 16 carbon atoms, and in particular C₈-C₁₆ branched alkanes, like C₈-C₁₆ isoalkanes of petroleum origin (also called isoparaffins) such as isododecane (also called 2,2,4,4,6-pentamethylheptane), isodecane, isohexadecane, and for example the oil sold under the trade names Isopars® or Permetyls® and the C₈-C₁₆ branched esters, such as isohexylneopentanoate and mixtures thereof.

Other volatile hydrocarbon oils such as petroleum distillates, in particular those sold under the name “Shell Soft®” by Shell, may also be used.

As volatile oils, volatile silicones may also be used, like for example volatile linear or cyclic silicone oils, in particular those having a viscosity of 6 centiStokes (6×10⁻⁶ m²/s) and having in particular from 3 to 6 silicon atoms, where these silicones could comprise one or more alkyl or alkoxy groups having 1 or 2 carbon atoms. The following can in particular be given as volatile silicon oil usable in the invention: octamethyl cyclotetrasiloxane, decamethyl cyclopentasiloxane, dodecamethyl cyclohexasiloxane, heptamethyl hexyltrisiloxane, heptamethyloctyl trisiloxane, hexamethyl disiloxane, octamethyl trisiloxane, decamethyl tetrasiloxane, dodecamethyl pentasiloxane and mixtures thereof.

Volatile organic solvents, in particular with fluorine, may also be used, such as nonafluoromethoxy butane or perfluoromethylcyclopentane.

Each of the compositions conforming to the invention may also comprise at least one nonvolatile organic oil or solvent, which may in particular be selected from nonvolatile hydrocarbon and/or silicon and/or fluorine oils.

The following can in particular be listed as nonvolatile hydrocarbon oils:
hydrocarbon oils of vegetable origin such as triglycerides made up of fatty acid esters and glycerol in which the fatty acids may have chain lengths varying from C4 to C24, where these chains may be linear or branched, saturated or unsaturated; these oils are in particular wheat germ, sunflower, grape seed, sesame, corn, apricot, castor, shea, avocado, olive, soy, sweet almond, palm, canola, cotton, hazelnut, Macadamia, jojoba, alfalfa, poppy, ambercup, sesame, squash, canola, blackcurrant, common evening primrose, millet, barley, quinoa, rye, safflower, candle nut tree, passion fruit, Muscat rose oil; or even triglycerides of caprylic/capric acids like those sold by Stéarineries Dubois are those sold under the name Miglyol 810®, 812® and 818® by Dynamit Nobel;
synthetic ethers with 10 to 40 carbon atoms;
linear or branched hydrocarbons of mineral or synthetic origin such as Vaseline, polydecanes, hydrogenated polyisobutene such as parléam, squalane and mixtures thereof;
synthetic esters like oils with formula R1COOR2 in which R1 represents a linear or branched fatty acid comprising 1 to 40 carbon atoms and R2 represents a hydrocarbon chain, in particular branched, containing 1 to 40 carbon atoms on the condition that R1+R2 is 10, like for example Purcellin oil (cetostearyl octanoate), isopropyl myristate, isopropyl palmitate, C₁₂ to C₁₅ alcohol benzoate, hexyl laurate, diisopropyl adipate, diisononyl isononanoate, 2-ethylhexyl palmitate, isostearate isostearate, octanoates, decanoates or ricinoleate of alcohols or polyalcohols like propyleneglycol dioctanoates; hydroxylated esters like isostearyl lactate, diisostearyl malate, and esters of pentaerythritol;
fatty alcohols, liquid at ambient temperature, with branched and/or unsaturated carbon chain having 12 to 26 carbon atoms like octyl dodecanol, isostearylic alcohol, oleic alcohol, 2-hexyldecanol, 2-butyloctanol, and 2-undecylpentadecanol;
higher fatty acids such as oleic acid, linoleic acid, linolenic acid;
   and mixtures thereof.

The nonvolatile silicon oils that can be used in one or the other of compositions (i) or (ii) conforming to the invention may be nonvolatile polydimethylsiloxanes (PDMS), polydimethylsiloxanes comprising alkyl or alkoxy groups, in the middle and/or the end of the silicon chain, groupings each having from 2 to 24 carbon atoms, phenylated silicones like phenyl trimethicones, phenyl dimethicones, phenyl trimethylsiloxy diphenylsiloxanes, diphenyl dimethicones, diphenyl methyldiphenyl trisiloxanes, 2-phenylethyl trimethylsiloxysilicates.

The fluorine oils which can be used in the compositions conforming to the invention are in particular fluorosilicone oils, fluoropolyethers, and fluorosilicones such as described in the document EP-A-847,752.

The concentration of non-volatile organic oil or solvent in the composition conforming to the invention goes from 0.01 to 30% by weight, in particular from 0.1 to 25% by weight and better from 0.1 to 20% by weight relative to the total weight of the composition.

Colorants

The compositions conforming to the invention may also comprise at least one colorant like powdered materials, liposoluble colorants and hydrosoluble colorants.

The powdered colorants may be selected from pigments and mother-of-pearl.

The pigments may be white or colored, mineral and/or organic, coated or not. The following may be listed among the mineral pigments: titanium dioxide, which could have a surface treatment, zirconium oxides, zinc or cerium, and also iron or chrome oxides, manganese violet, ultramarine blue, chrome hydrate and ferric blue. The following may be listed among the organic pigments: lamp black, type D & C pigments and the lakes based on cochineal carmine, barium, strontium, calcium and aluminum.

The mothers of pearl may be selected from white mother-of-pearl pigments such as mica covered with titanium or bismuth oxychloride, colored mother-of-pearl pigments such as titanium mica with iron oxides, titanium mica with in particular ferric blue or chrome oxide, titanium mica with an organic pigment of the aforementioned type and also mother-of-pearl pigments based on bismuth oxychloride.

The liposoluble colorants are for example Sudan red, D&C Red 17, D&C Green 6, β-carotene, soy oil, Sudan brown, D&C Yellow 11, D&C Violet 2, D&C Orange 5, quinoline yellow and annatto.

These colorants may be present at a concentration ranging from 0.01 to 30% by weight relative to the total weight of the composition.

Fillers

The compositions conforming to the invention may also comprise at least one filler.

The fillers may be selected from those well known to the person skilled in the art and commonly used in cosmetic compositions. The fillers may be mineral or organic, lamellar or spherical. The following can be mentioned: Mica, silica, kaolin, polyamide powders like Nylon® sold under the name Orgasol® by Atochem, poly-13-alanine and polyethylene, powders of tetrafluoroethylene polymers like Teflon®, lauroyl-lysine, starch, boron nitride, hollow expanded polymerized microspheres such as those of polyvinylidene/acrylonitrile chloride like those sold under the name Expancel® by Nobel Industrie, acrylic powders such as those sold under the name Polytrap® by Dow Corning, polymethylmethacrylate particles and silicone resin microbeads (Tospearls® from Toshiba, for example), precipitated calcium carbonate, magnesium carbonate and hydrocarbonate, hydroxyapatite, hollow silica microspheres (Silica Beads® from Maprecos), glass or ceramic microcapsules, and metallic soaps derived from carboxylic organic acids having from 8 to 22 carbon atoms, in particular from 12 to 18 carbon atoms, for example zinc, magnesium or lithium stearate, zinc laurate and magnesium myristate.

A compound may also be used which could swell under heating and in particular thermoexpandable particles such as non-expanded microspheres of vinylidene chloride/acrylonitrile/methylmethacrylate copolymer or of copolymer of homopolymer of acrylonitrile like for example those sold respectively under the names Expancel® 820 DU 40 and Expancel® 007WU by Akzo Nobel. The Advancell® sold by Sekuisi Plastic or the Thermoexpandable Microspheres® from Matsumoto can also be cited.

The fillers may represent from 0.1 to 25%, in particular from 0.2 to 20% by weight relative to the total weight of the composition.

Fibers

The compositions conforming to the invention may also comprise at least one fiber which provides an improvement of the lengthening effect.

“Fiber” must be understood to mean an object of length L and diameter D such that L is much larger than D, where D is the diameter of the circle in which the section of the fiber is inscribed. In particular, the ratio LID (form factor) is selected in the range extending from 3.5 to 2500, in particular from 5 to 500, and more specifically from 5 to 150.

The fibers which can be used in the composition from the invention may be fibers of synthetic or natural, mineral or organic origin. They may be short or long, isolated or organized in tresses for example, hollow or solid. The section thereof may be any shape and in particular circular or polygonal section (e.g. square, hexagonal or octagonal) according to the specific application considered. The shape thereof may be linear, curved, sinusoidal or curly. In particular, the ends thereof may be blunt and/or polished for avoiding injury.

In particular, the fibers have a length ranging from 1 μm to 10 mm, in particular from 0.1 mm to 5 mm and more particularly from 0.3 mm to 3.5 mm.

The section thereof may be included in a circle whose diameter ranges from 2 nm to 500 μm, in particular ranging from 100 nm to 100 μm and more particularly from 1 pm to 50 μm. The weight or count of fibers is often given in denier or decitex and represents the weight in grams for 9 km of thread. The fibers according to the invention may in particular have a count chosen in the range from 0.15 to 30 deniers and in particular from 0.18 to 18 deniers.

The fibers which can be used in the composition from the invention may be selected from stiff or non-stiff fibers; they may be of synthetic or natural origin, mineral or organic.

Further, the fibers may be surface treated or not, coated or not, and colored or not colored.

The following can be mentioned as fibers that can be used in the composition according to the invention: non-stiff fibers such as polyamide fibers (Nylon®) or stiff fibers such as polyamide-amide fibers like those sold under the name Kermel®, Kermel Tech® by Rhodia or of poly-(p-phenylene terephthalamide), or aramid, in particular sold under the name Kevlar® by DuPont de Nemours.

The fibers may be present in the composition according to the invention at a concentration ranging from 0.01% to 10% by weight relative to the total weight of the composition, in particular from 0.1% to 5% by weight, and more particularly from 0.3% to 3% by weight. The compositions conforming to the invention may also comprise at least one active cosmetic ingredient.

Active Cosmetic Ingredients

As cosmetic active ingredients that may be used in compositions conforming to the invention, the following may be mentioned in particular: antioxidants, preservatives, fragrances, neutralizers, emollients, thickeners, coalescing agents, plasticizers, hydrating agents, vitamins and filters, in particular solar, and mixtures thereof.

Of course, the person skilled in the art will take care to select the possible complementary additives and/or their quantity such that the advantageous properties of the composition according to the invention are not, or substantially not, altered by the intended addition.

By changing the quantity of waxes, thickeners, film-forming polymers and fillers, it is possible to get a makeup result that is satisfactory from the perspective of lengthening, curling and volume of the eyelashes.

### EXAMPLES

The following compositions were made. The quantities indicated are expressed as percentage by mass relative to the total weight of the composition.

Table 1 exemplifies a mascara formula based on hydroxypropyl starch phosphate, under the name Structure® XL from Akzo Noble.

This emulsion is used in the example A below at 4% and corresponds to the invention. Example B corresponds to the comparative formula using triethanolamine in order to assure the stability of the emulsion.

TABLE 1

| | Example A | Example B |
|---|---|---|
| INCI Name | (%) | (%) |
| Hydroxypropyl starch phosphate & water | 4.00 | — |
| Triethanolamine | — | 1.67 |
| Stearic acid & palmitic acid | — | 3.00 |
| C10-18 Triglycerides | 4.00 | 4.00 |
| Glyceryl behenate | 5.00 | 5.00 |
| Beeswax & benzyl alcohol & benzyl | 5.00 | 5.00 |
| cinnamate & benzyl benzoate | | |
| Glyceryl hydroxystearate | 2.50 | 2.50 |
| Jojoba esters & polyglycerin-3 & acacia | 4.50 | 4.50 |
| decurrens flower wax & helianthus | | |
| annuus seed wax | | |
| Octyldodecyl myristate | 2.00 | 2.00 |
| Sucrose acetate isobutyrate | 1.00 | 1.00 |
| Castor seed oil & hydrogenated castor oil | 2.00 | 2.00 |
| Water | 48.65 | 48.83 |
| Phenylpropanol & propanediol & caprylyl | 1.00 | 1.00 |
| glycol & tocopherol | | |
| Pullulan & sorbitol & acacia senegal | 5.00 | 5.00 |
| gum & trehalose | | |
| Glycerin | 5.00 | 5.00 |
| Tocopheryl acetate | 0.30 | 0.30 |
| Pigments Iron Oxides | 10 | 10 |
| TOTAL | 100.00 | 100.00 |

The formula B mascara is a conventional formula known from the state-of-the-art, used as control. This has a satisfactory consistency (in particular by the addition of “stearic acid & palmitic acid” which plays a thickening and co-emulsifying role in example B, making it possible to get a composition B the closest possible to example A in terms of texture) and the good dispersion of the waxes and pigments which form it. In that way a black, homogeneous product results which is easy to apply to the eyelashes and forms a buildable and smooth deposit that has a desired volume effect.

A similar effect can be obtained with the invention without using triethanolamine stearates or derivatives in order to form the emulsion for the composition.

The mascara formula from example A has a satisfactory consistency and a good dispersion of waxes and pigments which provides a black shade, such as desired for this type of product.

These mascaras apply easily to the eyelashes and form a smooth and homogeneous, buildable deposit and provide the desired volumizing result.

A stable wax-in-water emulsion mascara results that is free of triethanolamine or derivatives and has a makeup result that is entirely satisfactory and comparable to a mascara with triethanolamine or derivatives.

It is noted at the end of these various tests that Structure® XL made it possible not only to assure the stability of the wax-in-water emulsion but also to play on the texture of the formula and to get more or less elastic, more or less liquid emulsions, which represents a big advantage for the formulator.

Conversely, a formula emulsified with triethanolamine stearates needs a non-negligible quantity of additional texturizers in order to get a formula with sufficient consistency, but a formula emulsified with a hydroxyl starch requires little or no texturizer.

The makeup result of a cosmetic composition for eyelashes containing Structure® XL is the following: the eyelashes are enhanced over the full length.

1.-10. (canceled)

11. A cosmetic composition for coating keratinous fibers in the form of a wax-in-water emulsion comprising an emulsifying system, wherein said emulsifying system consists of a hydroxyalkyl starch and constitutes the only emulsifying system of the composition.

12. The composition according to claim 11, wherein the hydroxyalkyl starch is selected from hydroxyethyl starch, hydroxypropyl starch, and in particular hydroxypropyl starch phosphate and hydroxyethyl starch phosphate.

13. The composition according to claim 11, wherein the hydroxyalkyl starch is the hydroxypropyl starch phosphate.

14. The composition according to claim 11, wherein the hydroxyalkyl starch is present at a concentration ranging from 2% to 10% by weight, preferably from 4% to 8% by weight and better around 5% by weight relative to the total weight of the composition.

15. The composition according to claim 11, wherein the composition comprises an aqueous phase present at a concentration ranging from 1% to 95% by weight, relative to the total weight of the composition, preferably ranging from 3% to 80% by weight, and preferably ranging from 5% to 60% by weight.

16. The composition according to claim 11, wherein the wax is present at a concentration ranging from 0.1% to 50% by weight relative to the total weight of the composition, preferably from 1% to 40% by weight and more preferably from 5% to 30% by weight.

17. The composition according to claim 11, wherein the composition comprises at least one hydrophilic or lipophilic film-forming polymer.

18. The composition according to claim 11, wherein the composition comprises less than 1%, preferably less than 0.5% by weight of thickening compounds, and better, is free of thickening compounds.

19. The composition according to claim 11, wherein the composition comprises at least one additive selected from colorants, fillers, fibers, antioxidants, preservatives, fragrances, neutralizers, emollients, thickeners, coalescing agents, plasticizers, hydrating agents, vitamins and filters, in particular solar filters, and mixtures thereof.

20. A mascara cosmetic composition comprising hydroxypropyl starch phosphate as a single emulsifying system for the mascara cosmetic composition.
